(19) United States
(12) Patent Application Publication
(10) Pub. No.: US 2020/0330090 A1
(43) Pub. Date: Oct. 22, 2020

(54) Bidirectional guide suture device for pelvic floor reconstruction puncture

(71) Applicant: Lan Zhu, Beijing (CN)

(72) Inventor: Lan Zhu, Beijing (CN)

(21) Appl. No.: 16/920,455

(22) Filed: Jul. 3, 2020

(30) Foreign Application Priority Data
Jun. 15, 2020 (CN) 202010544542.0

(51) Int. Cl. A61B 17/04

(52) U.S. Cl. CPC A61B 17/0469; A61B 2017/06052; A61B 17/0482

(57) ABSTRACT

The present invention discloses a bidirectional guide suture device for pelvic floor reconstruction puncture, comprising a puncture catheter and a bidirectional guide cable. One end of the bidirectional guide cable is provided with a needle hole for connecting stitches, and the other end is provided with a pulling hole for pulling the bidirectional guide cable. The bidirectional guide cable is arranged inside the puncture catheter, and the pulling hole is located at the puncture end of the puncture catheter. The bidirectional guide suture device in the present invention is convenient and flexible in operation, can reduce puncture injury, and is especially used in pelvic floor reconstruction suture in human bodies.

### TECHNICAL FIELD

The present invention relates to the technical field of medical instruments, and more particularly relates to a bidirectional guide suture device for pelvic floor reconstruction puncture in pelvic floor reconstruction suture in human bodies.

### BACKGROUND

In the existing pelvic floor reconstruction suture surgery of the human body in the vaginal surgery, a metal guide puncture needle is used, and the front end of the guide puncture needle is provided with a sharp puncture head. The tail end has a needle hole. The guide puncture needle is provided with a guide protecting tube; the front, parts of the guide puncture needle and the protecting tube are curved into a circular arc, and the tail of the guide puncture needle can be inserted into the protecting tube.

The use method is as follows: the tail of the guide puncture needle is penetrated from the head end of the protecting tube; the puncture head extends from the front end; stitches penetrate through the needle hole on the tail of the guide puncture needle; after the protecting tube and the guide puncture needle which are sleeved together penetrate through the relevant part of the human body, the guide puncture needle is pulled out from the front part of the protecting tube, and the stitches also, reach the required part; then, the protecting tube is withdrawn from the human body; and the guide puncture needle is punctured and withdrawn again from other safe parts to complete one puncture and leading operation.

The disadvantages are as follows: 1. two puncture operations are required to for the guide puncture needle to guide the stitches once. 2. The guide puncture needle can only be pulled forward and not backward. In case of a special case, the puncture can be conducted only after the entire instrument is withdrawn. 3. The guide puncture needle is made of metal material and has a sharp front end. In the guide process, large penetration or pulling resistance makes it impossible to adjust a guide direction and, easy to injure other organ issues.

In conclusion, the existing guide needle is high in operation difficulty, large in patient injury and easy to cause secondary injury to the patient. Moreover, because the instrument is repeatedly cleaned and disinfected for use, the puncture head is easy to become blunt, thereby causing operation difficulty.

Therefore, the problem to be urgently solved by those skilled in the art is to research a bidirectional guide suture device for pelvic floor reconstruction puncture, which is convenient and flexible in operation and can reduce puncture injury.

### SUMMARY

In view of this, the present invention provides a bidirectional guide suture device for pelvic floor reconstruction puncture in pelvic floor reconstruction suture in human bodies, which is convenient and, flexible in operation and can reduce puncture injury.

To achieve the above purpose, the present invention adopts the following technical solution:

The bidirectional guide suture device for pelvic floor reconstruction puncture comprises:

a puncture catheter;

a bidirectional guide cable, wherein one end of the bidirectional guide cable is provided with a needle hole for connecting stitches, and the other end, is provided with a pulling hole for pulling the bidirectional guide cable; the bidirectional guide cable is arranged inside the puncture catheter, and the pulling hole is located at the puncture end of the puncture catheter.

The beneficial effects of adopting the above technical solution are: in the present invention, one puncture and leading operation can be completed by one puncture of the puncture catheter; and moreover, no sharp needle exists at both ends of the bidirectional guide cable, so that other injury may not be caused to the human body during the puncture.

Preferably, the puncture end of the puncture catheter is provided with a beveled cutting edge, which is convenient for puncture for placing the puncture catheter into the body.

Preferably, the beveled cutting edge is a single-sided cutting edge or a double-sided cutting edge.

Preferably, the puncture end of the puncture catheter is curved, and curved into a circular arc.

Preferably, one end of the puncture catheter away from the puncture end is connected with a handle; a through hole is formed inside the handle; and the puncture catheter is sleeved inside the through hole. In use, the puncture catheter is placed into the body by holding the handle; the puncture catheter is penetrated inside the handle, and pierces the handle, so that the bidirectional guide cable can enter from the front end of the puncture catheter or enter from the tail of the puncture catheter.

Preferably, the outer diameters of the bidirectional guide cable, the pulling hole and a needle hole are smaller than the inner diameter of the puncture catheter, so that the bidirectional guide cable can move more freely in the puncture catheter and the through hole.

Preferably, the bidirectional guide cable can be bent and is molded by semi-flexible polymer plastic; and the bending performance of the bidirectional guide cable allows the bidirectional guide cable to be taken out from the puncture catheter, enter the puncture catheter from the puncture end of the puncture catheter after complete leading, and to be taken out from the puncture catheter.

It can be known form the above technical solution that compared with the prior art, the present invention provides a bidirectional guide suture device for pelvic floor reconstruction puncture, which has the following beneficial effects:

(1) In the present invention, the bidirectional guide cable can achieve bidirectional guide suture, reduce the puncture operation, reduce the injury to patients, and reduce the risk of secondary injury to other organ issues during guide.

(2) Meanwhile, the present invention is convenient to operate, simple, low in resistance, flexible to use, can adjust the angle and posture arbitrarily also avoids cross, infection, and has low use cost.

### DESCRIPTION OF DRAWINGS

To more clearly describe the technical solution in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a structural schematic diagram of a puncture catheter provided by the present invention; and

FIG. 2 is a structural schematic diagram of a bidirectional guide cable provided by the present invention.

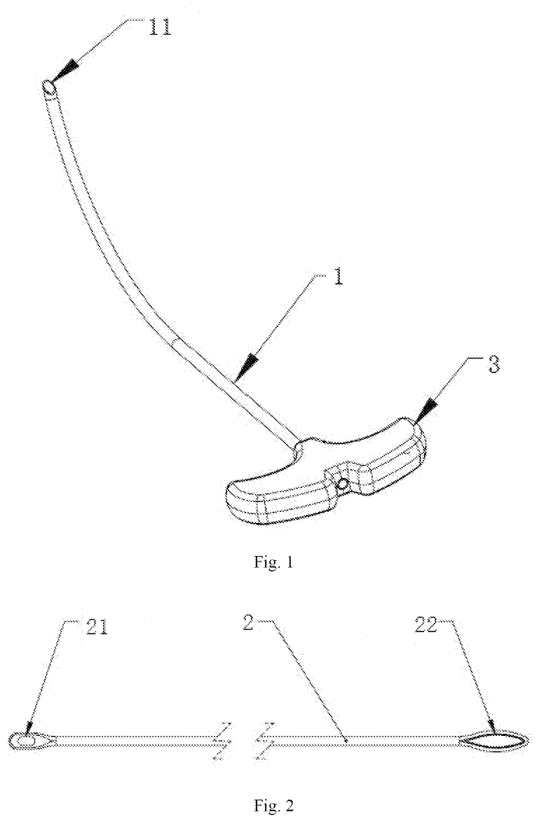

In the figures,
1—puncture catheter.
   11—beveled cutting edge;
2—bidirectional guide cable;
   21—needle hole; 22—pulling hole;
3—handle.

### DETAILED DESCRIPTION

The technical solution in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Embodiments of the present invention disclose a bidirectional guide suture device for pelvic floor reconstruction puncture, comprising:

a puncture catheter 1;

a bidirectional guide cable 2, wherein, one end of the bidirectional guide cable 2 is provided with a needle hole 21 for connecting stitches, and the other end is provided with a pulling hole 22 for pulling the bidirectional guide cable 2; the bidirectional guide cable 2 is arranged inside the puncture catheter 1, and the pulling hole 22 is located at the puncture end of the puncture catheter 1.

To further optimize the above technical solution, the puncture end of the puncture catheter 1 is provided with a beveled cutting edge 11.

To further optimize the above technical solution, the beveled cutting edge 11 is a single-sided cutting edge or a double-sided cutting edge.

To further optimize the above technical solution, the puncture end of the puncture catheter 1 is curved, and curved into a circular arc.

To further optimize the above technical solution, one end of the puncture catheter 1 away from the puncture end is connected with a handle 3; a through hole is formed inside the handle 3; and the puncture catheter 1 is sleeved inside the through hole.

To further optimize the above technical solution, the outer diameters of the bidirectional guide cable 2, the pulling hole 22 and a needle hole 21 are smaller than the inner diameter of the puncture catheter 1. The bidirectional guide cable 2 can be flexibly penetrated or withdrawn from both ends of the puncture catheter 1, does not cause the risk of secondary injury to other organ issues during guide, and can increase a surgical space. The resistance during penetration or withdrawal is small, and the puncture catheter 1 can adjust the angle and the posture arbitrarily, thereby having the advantages of convenient and simple operation and reduction of patient pain.

To further optimize the above technical solution, the bidirectional guide cable 2 can be bent and is molded by semi-flexible polymer plastic.

The operation process is as follows:

In use during the surgery, after the handle 3 is held to puncture the puncture catheter 1 into the relevant part of the human body, stitches are penetrated into the needle hole 21 of the bidirectional guide cable 2 and the bidirectional guide cable 2 is placed into the puncture catheter 1; the pulling hole 22 for pulling the bidirectional guide cable 2 pulls out the bidirectional guide cable from the puncture end of the puncture catheter 1, and the stitches also penetrate through the puncture catheter 1 along with the bidirectional guide cable 2 to reach a required part; after the stitches are removed, the bidirectional, guide cable 2 is bent in the surgical space, penetrates from the puncture end of the puncture catheter 1 into the puncture catheter 1, and is withdrawn from the body; and finally, the puncture catheter 1 is withdrawn from the body, and one puncture and leading operation can be completed by one puncture. The stitches penetrated into the human body are used to connect synthetic meshes to complete the surgery of female pelvic organ prolapse.

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention, Therefore, the present invention will not be limited to these embodiments shown herein, but will conform to the widest scope consistent with the principle and novel features disclosed herein.

1. A bidirectional guide suture device for pelvic floor reconstruction puncture, comprising:
a puncture catheter (1);
a bidirectional guide cable (2), wherein one end of the bidirectional guide cable (2) is provided with a needle hole (21) for connecting stitches, and the other end is provided with a pulling hole (22) for pulling the bidirectional guide cable (2); the bidirectional guide cable (2) is arranged inside the puncture catheter (1), and the pulling hole (22) is located at the puncture end of the puncture catheter (1).

2. The bidirectional guide suture device for pelvic floor reconstruction puncture according to claim 1, wherein the puncture end of the puncture catheter (1) is provided with a beveled cutting edge (11).

3. The bidirectional guide suture device for pelvic floor reconstruction puncture according to claim 2, wherein the beveled cutting edge (11) is a single-sided cutting edge or a double-sided cutting edge.

4. The bidirectional guide suture device for pelvic floor reconstruction puncture according to claim 1, wherein the puncture end of the puncture catheter (1) is curved, and curved into a circular arc.

5. The bidirectional guide suture device for pelvic floor reconstruction puncture according to claim 4, wherein one end of the puncture catheter (1) away from the puncture end is connected with a handle (3); a through hole is formed inside the handle (3); and the puncture catheter (1) is sleeved inside the through hole.

6. The bidirectional guide suture device for pelvic floor reconstruction puncture according to claim 5, wherein the outer diameters of the bidirectional guide cable (2), the pulling hole (22) and a needle hole (21) are smaller than the inner diameter of the puncture catheter (1).

7. The bidirectional guide suture device for pelvic floor reconstruction puncture according to claim 6, wherein the bidirectional guide cable (2) can be bent and is molded by semi-flexible polymer plastic.
